SEQUENCE LISTING (9 sequences)

SEQ ID NO: 1 - DNA, 17 bp, Artificial
  chemically synthesized
ccgtggtcatgctccaa

SEQ ID NO: 2 - DNA, 32 bp, Artificial
  chemically synthesized
gagattagtactgaagagttggagcatgacca

SEQ ID NO: 3 - RNA, 35 nt, Artificial
  chemically synthesized
gagauuaguacugaagaguuggagcaugaccacgg

SEQ ID NO: 4 - RNA, 20 nt, Artificial
  chemically synthesized
cucuucaguacuaaucucuu

SEQ ID NO: 5 - DNA, 17 bp, Artificial
  chemically synthesized
aaccgtggtcatgctcc

SEQ ID NO: 6 - protein, 12 aa, Artificial
  chemically synthesized
ACDCRGDCFCGG

SEQ ID NO: 7 - DNA, 15 bp, Artificial
  chemically synthesized
ccgtggtcatgctcc

SEQ ID NO: 8 - RNA, 36 nt, Artificial
  chemically synthesized
cguacgcggaauacuucgauuggagcaugaccacgg

SEQ ID NO: 9 - RNA, 21 nt, Artificial
  chemically synthesized
ucgaaguauuccgcguacguu

(19) United States
(12) Patent Application Publication
(10) Pub. No.: US 2009/0258424 A1
(43) Pub. Date: Oct. 15, 2009

(54) Cellular Delivery of siRNA

(75) Inventor: Yingqun Huang, East Haven, CT (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: Yale University, New Haven, CT (US)

(21) Appl. No.: 12/280,468

(22) PCT Filed: Mar. 1, 2007
(86) PCT No.: PCT/US07/05423

§ 371 Date: Aug. 22, 2008

Related U.S. Application Data
(60) Provisional application No. 60/778,135, filed on Mar. 1, 2006.

(51) Int. Cl. C12N 15/87; C12N 5/06; C07H 21/02; C07K 16/00; C07K 14/62

(52) U.S. Cl. 435/455; 435/375; 536/24.5; 530/394; 530/399; 530/303

(57) ABSTRACT

The invention provides a method for delivering a nucleic acid to a cell using a targeting molecule that is bound non-covalently to the nucleic acid. Compositions and kits are also provided.

### BACKGROUND OF THE INVENTION

The RNAi phenomenon was first discovered in Caenorhabditis elegans when exogenously introduced long double-stranded RNAs (dsRNAs) were found to silence the expression of a target gene much more efficiently than either strand alone (Fire et al., 1998, Nature 392:806-811). This phenomenon has been subsequently observed in many other organisms, including mammals (reviewed in Zamore, 2002 Science 296:1265-1269 and Dykxhoom et al., 2003, Nat Rev Mol Cell Biol. 4(6): 457-67). Thus, RNAi appears to be an ancient and general mechanism for gene regulation and might have evolved to have both developmental and antiviral roles. Intensive biochemical and genetic studies on RNAi have been carried out, and a great deal of its mechanistic basis has been uncovered. In the cytoplasm, long dsRNAs are cut by the cellular endonuclease Dicer into duplexes of ˜22-nt called small interfering RNAs (siRNAs) that lead to the cleavage and degradation of target mRNAs bearing perfect complementarity.

The high specificity and potency of RNAi makes it an especially attractive tool in gene therapy. Since every cell has the necessary machinery, RNAi can in principle be used to inhibit any gene, including those that are resistant to conventional therapy. Like other forms of gene therapy, however, the application of RNAi therapeutics faces two major obstacles: cell and tissue type-specific delivery and stability of the reagents to be delivered. When naked siRNAs are used, they suffer from the problem of poor cell membrane-penetration capability and short blood circulation half-life due to their high susceptibility to nuclease degradation, as well as rapid filtration by the kidney owing to their small size (˜15 kDa). It is possible to chemically modify siRNAs to make them resistant to nucleases without compromising biological activity (Soutschek et al., 2004, Nature 432:173-178). Also, by coupling siRNAs to other molecules such as peptides, antibodies, or other cell surface receptor ligands, or encasing them in liposomes or other types of particles, it is possible to produce siRNA drugs that have prolonged lifetimes in animals, and that can be delivered to specific cells and tissues (reviewed in Oliveira et al., 2006, J Biomed Biotechnol. 2006: Article ID 63675, 1-9).

A variety of molecules have been used for cell-specific siRNA delivery. For example, the nucleic acid-condensing property of protamine has been combined with specific antibodies to deliver siRNAs (Song et al., 2005, Nat. Biotch. 23:709-717). A recombinant protein composed of the protamine coding region fused to the C-terminus of the heavy chain Fab fragment of an ErbB2 antibody was used to assemble protein-siRNA complexes. These complexes were protected from nuclease degradation and able to induce specific gene silencing when systemically delivered to ErbB2-expressing cancer cells in a mouse model (Song et al., 2005, Nat. Biotech. 23:709-707). The self-assembly PEGylated polycation polyethylenimine (PEI) has also been used to condense and protect siRNAs (Schiffelers et al., 2004, Nuc Acids Res. 32:e149, 141-110). Decorated on the surface of the formulated nanoparticles were Arg-Gly-Asp (RGD)-containing peptides that have high affinity for integrins. Integrins are overexpressed on the surface of angiogenic endothelial cells of tumor neovasculature and of many cancer cells. The siRNA-containing nanoparticles were then successfully delivered to integrin-overexpressing tumor neovasculature, leading to inhibition of vascular endothelial growth factor receptor-2 (VEGF R2) expression and thereby tumor angiogenesis. In another study, siRNAs was encapsulated in cyclodextrin-containing polycation particles that had transferrin as a targeting ligand for delivery to transferrin receptor-expressing tumor cells (Hu-Lieskovan et al., 2005, Cancer Res 65:8984-8992). Systemic delivery of these siRNAs against the EWS-FLI1 gene product resulted in dramatic inhibition of tumor growth in a murine model of metastatic Ewing's sarcoma.

Recently, high affinity and specificity nucleic acid aptamers capable of binding cell surface antigens and subsequently being internalized have become increasingly attractive alternatives to antibodies for siRNA delivery. At least one such aptamer, the aptamer for the prostate-specific membrane antigen (PSMA), has been shown to carry siRNA-containing complexes into antigen-expressing cells (Chu et al., 2006, Nuc Acids Res. 34:e73, 71-76). When non-covalently coupled to siRNAs via a streptavidin bridge, the aptamer:streptavidin:siRNA complexes were efficiently and specifically taken up by antigen-expressing prostate cancer cells and resulted in targeted gene silencing. Using a different strategy, aptamer-siRNA chimeras were constructed through sequence extension of the aptamer (McNamara et al., 2006, Nat. Biotch. 24:1005-1015). The chimeras were able to silence two tumor-related genes in addition to reducing cellular proliferation and inducing apoptosis in PSMA-expressing cells.

Although they demonstrate both specific and efficient siRNA delivery and function, the above-mentioned approaches disadvantageously require complex procedures to make the desired complexes or particles. These complex procedures pose potential disadvantages to the eventual development and application of the methods. In the case of the streptavidin-based method, which involves a simpler chemistry, the feasibility may be limited by the likelihood of immune response induced by streptavidin (Breitz et al., 2000, J Nucl Med. 41:131-140). Furthermore, in strategies using high affinity receptors, release of a receptor-bound molecule may be poor. For instance, it has been observed that molecules conjugated to folate are not necessarily released from the folate receptor after cellular internalization and are subsequently trafficked back to the plasma membrane (Yang et al., 2006, PNAS Epub 2006 Sep. 1).

Thus, there is a need in the art for a method of delivering siRNA specifically and efficiently. This invention addresses that need.

### BRIEF SUMMARY OF THE INVENTION

The invention provides a method for delivering a nucleic acid to cell comprising the steps of binding non-covalently a targeting molecule to a nucleic acid to form a targeted nucleic acid; and contacting the cell with the targeted nucleic acid; wherein i) the targeting molecule comprises a receptor-binding moiety linked to a first oligonucleotide comprising a hook sequence; ii) the nucleic acid comprises a sequence which is complementary to the hook sequence; iii) the cell comprises a cognate receptor for the receptor-binding moiety; and iv) the hook sequence is not a naturally-occurring sequence in the cell; wherein when the targeted nucleic acid binds to the cognate receptor on the cell, the targeted nucleic acid is taken up by the cell. In one embodiment, then nucleic acid is selected from the group consisting of dsDNA, antisense, antagomir, siRNA, ribozyme, and miRNA.

The invention further provides a method for delivering an RNA molecule to a cell, the method comprising the steps of binding a targeting molecule to the RNA molecule to form a targeted RNA molecule; and contacting the cell with the targeted RNA molecule; wherein i) the targeting molecule comprises a receptor-binding moiety linked to a first oligonucleotide comprising a hook sequence; ii) the RNA molecule comprises a sequence which is complementary to the hook sequence; and iii) the cell comprises a cognate receptor for the receptor-binding moiety; wherein when the targeted RNA molecule binds to the cognate receptor on the cell, the targeted RNA molecule is taken up by the cell. In some embodiments, the RNA molecule is selected from the group consisting of siRNA, ribozyme, and miRNA. In some embodiments, the hook sequence is not a naturally-occurring sequence in said cell.

In some embodiments of the methods of the invention, the receptor-binding moiety is selected from the group consisting of folate, an RGD-containing peptide or derivative or peptidomimetics thereof, epidermal growth factor (EGR), transferrin, low density lipoprotein (LDL), insulin, protein hormones, galactosamine, galactose, biotin, platelet-derived growth factor, thyrotrypsin releasing hormone (TRH), nerve-growth factor (NGF), Ct₂-macroglobulin, thiodothyronin, thrombine, arachidonic acid, transforming growth factor-α (TGF-α), a heregulin and alpha fetoprotein (AFP). In preferred embodiments of the methods of the invention, the receptor-binding moiety is selected from the group consisting of folate and an RGD-containing peptide or derivative or peptidomimetics thereof.

In some embodiments, the cell is a human cell.

Furthermore, the invention includes a method for delivering an siRNA molecule to a cell comprising the steps of binding a targeting molecule to the siRNA molecule to form a targeted siRNA molecule; and contacting the cell with the targeted RNA molecule; wherein i) the targeting molecule comprises a receptor-binding moiety linked to a first oligonucleotide comprising a hook sequence, wherein the receptor binding moiety is selected from the group consisting of folate and an RGD-containing peptide or derivative or peptidomimetics thereof; ii) the siRNA molecule comprises a sequence which is complementary to the hook sequence; and iii) the cell comprises a cognate receptor for the receptor-binding moiety; wherein when the targeted siRNA molecule binds to the cognate receptor on the cell, the targeted siRNA molecule is taken up by the cell. In one embodiment, the siRNA molecule is targeted to a gene selected from the group consisting of a gene whose product contributes to the development of chemoresistance and a gene whose product represses apoptosis. In another embodiment the cell is a human cell. In yet another embodiment, the hook sequence is not a naturally-occurring sequence in the cell.

In one embodiment of the method for delivering an siRNA molecule to a cell, the receptor-binding moiety is folate. In one aspect of this embodiment, the cell is a human nasopharyngeal carcinoma cell or a human epithelium ovarian carinoma cell.

In another embodiment of the method for delivering an siRNA molecule to a cell, the receptor-binding moiety is an RGD-containing peptide or derivative or peptidomimetics thereof. In one aspect of this embodiment, the cell is an endothelial cell of neovasculature or a cancer.

The invention further provides compositions and kits useful in practicing the methods of the invention.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIGS. 1A to 1D are a series of images illustrating the non-covalent tethering strategy of the invention. FIG. 1A depicts an oligonucleotide duplex (F+) comprising a folate-conjugated strand (solid circle; SEQ ID NO: 1) and a fluoroscein-labeled strand (star; SEQ ID NO. 2). FIG. 1B depicts an oligonucleotide duplex (F−) comprising the same fluoroscein-labeled strand (SEQ ID NO. 2; star) but lacking a folate conjugated to SEQ ID NO. 1. FIGS. 1C and 1D are fluorescent images of KB cells following treatment with F+ and F−, respectively.

FIG. 2 is a schematic illustration of targeted delivery of dsRNA to cells. Both strands of dsRNA have a targeting molecule (solid circle represents receptor-binding moiety) tethered to it by basepairing.

FIG. 3 depicts a bar graph illustrating results of real-time PCR of relative RNA levels in cells treated with a dsRNA targeted to the MyD88 mRNA under various conditions. The RNA levels of the targeted mRNA (MyD88, dark gray bars) and an untargeted mRNA (XIAP, light gray bars) from cells incubated with ODN/dsRNA were arbitrarily set as 1. The internal control RNA used for normalization in real-time PCR was 18S rRNA. Numbers were derived from two independent experiments. ODN/dsRNA: cells treated with dsRNA tethered to ODN not conjugated to folate. F+ODN/dsRNA: cells treated with dsRNA tethered to folate-conjugated ODN. F+ODN/dsRNA plus free folate: cells treated with F+ODN/dsRNA in the presence of 1000-fold molar excess of free folate.

FIG. 4 depicts a bar graph illustrating results of real-time PCR of relative RNA levels in cells treated with a dsRNA targeted to XIAP mRNA under various conditions. The RNA levels of the targeted mRNA (XIAP, black bar) and an untargeted mRNA (survivin, white bar) from cells incubated with ODN/dsRNA were arbitrarily set as 1. The internal control RNA used for normalization in real-time PCR was PBGD mRNA. Numbers were derived from three independent experiments.

FIGS. 5A and 5B are a series of images of a schematic of a tethered siRNA and of Western blots of cells after administration of a tethered siRNA. FIG. 5A depicts a schematic tethered siRNA comprising a sense strand (SEQ ID NO. 3) base-paired to both SEQ ID Nos. 4 and 5. The targeting molecule comprises SEQ ID NO. 5 conjugated to folate (black circle). After processing by Dicer, the sense strand is expected to comprise nucleotides 1-20 of SEQ ID No. 3. FIG. 5B is a series of images of Western blots of cellular extracts after 24 and 48 hours of incubation with various nucleic acid complexes, as shown schematically above each lane. The top panels are blots for XIAP protein levels. The bottom panels are blots for beta-actin, which was used as a loading control. Lanes 1 and 4 are F+ODN (folate-conjugated targeting molecule alone). Lanes 2 and 5 are F+ODN/XIAP. Lanes 3 and 6 are ODN/XIAP (siRNA comprising the oligonucleotide of the targeting molecule without the conjugated folate).

FIGS. 6A and 6B are a series of images of a schematic of a tethered siRNA and a bar graph illustrating extent of cell viability in chemoresistant human EOC cells R182 treated with a dsRNA targeted to XIAP mRNA under various conditions. FIG. 6A depicts a schematic tethered siRNA comprising a sense strand (SEQ ID NO. 3) base-paired to both SEQ ID Nos. 4 and 5. The targeting molecule comprises SEQ ID NO. 5 conjugated to folate (black circle). After processing by Dicer, the sense strand is expected to comprise nucleotides 1-20 of SEQ ID No. 3. FIG. 6B is a bar graph of the cell viability in EOC cells administered buffer, F+ODN/XIAP or ODN/XIAP (not conjugated to folate) and subsequently treated with taxol (taxol) or not treated with taxol (NT).

FIGS. 7A and 7B are a series of images of a schematic of a tethered siRNA and a bar graph illustrating results of real-time PCR of relative RNA levels in chemoresistant human EOC cells R182 treated with a dsRNA targeted to survivin mRNA under various conditions. FIG. 7A depicts a schematic tethered siRNA targeting survivin mRNA comprising a sense strand (SEQ ID NO. 8) base-paired to both SEQ ID Nos. 9 and 5. The targeting molecule comprises SEQ ID NO. 5 conjugated to folate (black circle). After processing by Dicer, the sense strand is expected to comprise nucleotides 1-21 of SEQ ID No. 8. FIG. 7B is a bar graph of the cell viability in EOC cells administered buffer, F+ODN/survivin or ODN/survivin (not conjugated to folate) and subsequently treated with taxol (taxol) or not treated with taxol (NT).

### DETAILED DESCRIPTION OF THE INVENTION

The invention provides a method of delivering a nucleic acid to a cell. The method employs a targeting molecule comprising an oligonucleotide conjugated to a receptor-binding moiety. The oligonucleotide component comprises a sequence that hybridizes to a substantially complementary sequence that is introduced into a nucleic acid to be delivered. The nucleic acid is thus targeted to a receptor of a cell by means of a non-covalently bound targeting molecule. The invention is useful by virtue of the fact that it provides a simple and efficient method for delivery of one or more nucleic acids to a cell.

### DEFINITIONS

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and nucleic acid chemistry and hybridization are those well known and commonly employed in the art.

Standard techniques are used for nucleic acid and peptide synthesis. The techniques and procedures are generally performed according to conventional methods in the art and various general references (e.g., Sambrook and Russell, 2001, Molecular Cloning, A Laboratory Approach, Cold Spring Harbor Press, Cold Spring Harbor, N.Y., and Ausubel et al., 2002, Current Protocols in Molecular Biology, John Wiley & Sons, NY), which are provided throughout this document.

The nomenclature used herein and the laboratory procedures used in analytical chemistry and organic syntheses described below are those well known and commonly employed in the art. Standard techniques, or modifications thereof, are used for chemical syntheses and chemical analyses.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

It is understood that any and all whole or partial integers between any ranges set forth herein are included herein.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. The antibodies useful in the present invention may exist in a variety of forms including, for example, polyclonal antibodies, monoclonal antibodies, intracellular antibodies (“intrabodies”), Fv, Fab and F(ab)₂, as well as single chain antibodies (scFv), camelid antibodies and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, are reduced.

By the term “applicator,” as the term is used herein, is meant any device including, but not limited to, a hypodermic syringe, a pipette, and the like, for administering the compounds and compositions of the invention.

The terms “effective amount” and “pharmaceutically effective amount” refer to a nontoxic but sufficient amount of an agent to provide the desired biological result. That result can be reduction and/or alleviation of the signs, symptoms, or causes of a disease, or any other desired alteration of a biological system. An appropriate effective amount in any individual case may be determined by one of ordinary skill in the art using routine experimentation.

“Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are substantially complementary to each other when at least 50% of corresponding positions in each of the molecules are occupied by nucleotides which normally base pair with each other (e.g., A:T and G:C nucleotide pairs).

A first region of an oligonucleotide “flanks” a second region of the oligonucleotide if the two regions are adjacent one another or if the two regions are separated by no more than about 500 nucleotide residues, and preferably no more than about 100 nucleotide residues and, more preferably, no more than about 10 nucleotide residues.

“Homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

“Instructional material,” as that term is used herein, includes a publication, a recording, a diagram, or any other medium of expression which can be used to communicate the usefulness of the composition and/or compound of the invention in a kit. The instructional material of the kit may, for example, be affixed to a container that contains the compound and/or composition of the invention or be shipped together with a container which contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

“Naturally-occurring” as applied to an object refers to the fact that the object can be found in nature. For example, a polypeptide or polynucleotide sequence that is present in an organism (including viruses) that can be isolated from a source in nature and which has not been intentionally modified by man is a naturally-occurring sequence.

In the context of the present invention, the following abbreviations for the commonly occurring nucleic acid bases are used. “A” refers to adenosine, “C” refers to cytidine, “G” refers to guanosine, “T” refers to thymidine, and “U” refers to uridine.

A “polynucleotide” means a single strand or parallel and anti-parallel strands of a nucleic acid. Thus, a polynucleotide may be either a single-stranded or a double-stranded nucleic acid.

The term “oligonucleotide” typically refers to short polynucleotides, generally no greater than about 60 nucleotides. It will be understood that when a nucleotide sequence is represented by a DNA sequence (i.e., A, T, G, C), this also includes an RNA sequence (i.e., A, U, G, C) in which “U” replaces “T.”

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). The term “nucleic acid” typically refers to large polynucleotides.

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

By “expression cassette” is meant a nucleic acid molecule comprising a coding sequence operably linked to promoter/regulatory sequences necessary for transcription and, optionally, translation of the coding sequence.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a n inducible manner.

An “inducible” promoter is a nucleotide sequence which, when operably linked with a polynucleotide which encodes or specifies a gene product, causes the gene product to be produced substantially only when an inducer which corresponds to the promoter is present.

“Polypeptide” refers to a polymer composed of amino acid residues, related naturally occurring structural variants, and synthetic non-naturally occurring analogs thereof linked via peptide bonds. Synthetic polypeptides can be synthesized, for example, using an automated polypeptide synthesizer.

The term “protein” typically refers to large polypeptides.

The term “peptide” typically refers to short polypeptides.

As used herein, an “RGD-containing” peptide is a peptide having a sequence which comprises RGD. Such peptides may be cyclic or otherwise modified to improve in vivo stability and activity.

Conventional notation is used herein to portray polypeptide sequences: the left-hand end of a polypeptide sequence is the amino-terminus; the right-hand end of a polypeptide sequence is the carboxyl-terminus.

As used herein, a “peptidomimetic” is a compound containing non-peptidic structural elements that is capable of mimicking the biological action of a parent peptide. A peptidomimetic may or may not comprise peptide bonds.

“Ribozymes” as used herein are RNA molecules possessing the ability to specifically cleave other single-stranded RNA in a manner analogous to DNA restriction endonucleases. Through the modification of nucleotide sequences encoding these RNAs, molecules can be engineered to recognize specific nucleotide sequences in an RNA molecule and cleave it (Cech, 1988, J. Amer. Med. Assn. 260:3030). There are two basic types of ribozymes, namely, tetrahymena-type (Hasselhoff, 1988, Nature 334:585) and hammerhead-type. Tetrahymena-type ribozymes recognize sequences which are four bases in length, while hammerhead-type ribozymes recognize base sequences 11-18 bases in length. The longer the sequence, the greater the likelihood that the sequence will occur exclusively in the target mRNA species. Consequently, hammerhead-type ribozymes are preferable to tetrahymena-type ribozymes for inactivating specific mRNA species, and 18-base recognition sequences are preferable to shorter recognition sequences which may occur randomly within various unrelated mRNA molecules. Ribozymes and their use for inhibiting gene expression are also well known in the art (see, e.g., Cech et al., 1992, J. Biol. Chem. 267:17479-17482; Hampel et al., 1989, Biochemistry 28:4929-4933; Eckstein et al., International Publication No. WO 92/07065; Altman et al., U.S. Pat. No. 5,168,053).

As used herein, a “receptor-binding moiety” refers to a molecule that binds specifically to a receptor. A receptor-binding moiety may be a naturally-occurring ligand for the receptor or functional derivatives thereof. A receptor-binding moiety may also be a small molecule mimetic of a naturally-occurring ligand, an antibody, a peptidomimetic, an aptamer or any other molecule provided it binds specifically to the receptor and induces receptor-mediated endocytosis of the bound moiety.

As used herein, a “cognate receptor” refers to the receptor to which a given receptor-binding moiety specifically binds. For instance, the folate receptor is the cognate receptor for folate.

As used herein, a “targeted nucleic acid” is a nucleic acid that is bound by sequence-specific base-pairing hydrogen bonds to a targeting molecule.

As used herein, a “targeting molecule” refers to a molecule comprising an oligonucleotide molecule covalently conjugated to a receptor-binding moiety. The oligonucleotide molecule comprises a hook complement sequence which is complementary to a hook sequence in a second oligonucleotide or nucleic acid molecule.

As used herein, “conjugated” refers to covalent attachment of one molecule to a second molecule.

As used herein, a “hook sequence” refers to a nucleotide sequence in a nucleic acid that is substantially complementary to a hook complement sequence in a targeting molecule.

As used herein, a “hook complement sequence” refers to a nucleotide sequence that is substantially complementary to a hook sequence.

### DESCRIPTION

The invention is a method of delivering a nucleic acid of interest to a cell using a targeting molecule comprising a receptor-binding moiety conjugated to an oligonucleotide. The nucleic acid of interest is designed or modified to comprise a hook sequence which is substantially complementary to a sequence of the targeting molecule. The oligonucleotide component of the targeting molecule is bound to the nucleic acid to be targeted by Watson-Crick-type hydrogen bonds between the nucleotides of the hook sequence and the nucleotides of the hook complement sequence. A cell comprising a cognate receptor for the receptor binding moiety is contacted with the targeted nucleic acid. The receptor binding moiety of the targeted nucleic acid binds to its cognate receptor on the cell and receptor-mediated endocytosis internalizes the targeted nucleic acid, thereby delivering the nucleic acid to the cell. With wishing to be bound by theory, it is believed that an endogenous cellular nuclease, such as RNase H or Dicer, or a helicase releases the targeted nucleic acid from the targeting oligonucleotide molecule.

The method is advantageous in providing an economical, rapid and easily adaptable solution to targeting cellular uptake of a nucleic acid of interest. In prior art methods of targeting, a targeting ligand is conjugated directly to a nucleic acid to be delivered. These methods are problematic in several regards, including inefficient release of a receptor-bound nucleic acid within a cell and the need for post-conjugational purification for each individual conjugate. In other prior art methods, a targeting ligand is conjugated to a polycation, such as poly-L-lysine or PEI, which binds non-specifically through electrostatic interactions to a nucleic acid. The problems associated with this strategy include controlling the stoichiometry of binding to the nucleic acid, complicated purification procedures and cytotoxicity of polycations. In contrast, in the inventive method, a single type of targeting molecule can be used to direct any nucleic acid of interest, designed or modified to comprise a hook sequence, to a cell comprising a cognate receptor for the receptor-binding moiety. Thus, the problems associated with conjugating a targeting ligand individually to each nucleic acid whose cellular delivery is desired are avoided. Furthermore, the invention overcomes other prior art issues, including poor release of a receptor-bound nucleic acid within a cell. Without wishing to be bound by theory, is it believed that the non-covalent tethering of a targeting molecule by base-pairing to a nucleic acid to be delivered contributes to this unexpected benefit of the invention. In addition, the double-stranded duplex resulting from binding of a targeting oligonucleotide to a hook sequence is believed to contribute to the in vivo stability and nuclease resistance of the nucleic acid, which may contribute favorably to a longer half-life of the targeted nucleic acid in vivo. Additionally, the length of the targeted nucleic acid can be easily adjusted to optimize in vivo delivery.

The method of the invention employs a targeting molecule which comprises an oligonucleotide conjugated to a receptor-binding moiety. The oligonucleotide component, which tethers the targeting molecule to a nucleic acid of interest, comprises a sequence called the “hook complement” that is substantially complementary to a hook sequence, which is the sequence that is introduced into a nucleic acid of interest that is to be targeted to a cell. Preferably the hook complement sequence is 100% complementary to the hook sequence, however, complementarity can be less than 100%, provided the hybridization between the hook sequence and the oligonucleotide sequence is sufficiently long-lived for the targeted nucleic acid to remain bound the targeting molecule to reach, bind and be internalized by the target cell. Determination of binding free energies for nucleic acid molecules is well known in the art (see, e.g., Turner et al., 1987, CSH Symp. Quant. Biol. LII pp. 123-133; Freier et al., 1986, Proc. Nat. Acad. Sci. USA 83:9373-9377; Turner et al., 1987, J. Am. Chem. Soc. 109:3783-3785; Chavali et al., 2005, Bioinformatics 21(20):3918-3925). In general, the length of the hook sequence or hook complement sequence is from about 12 nucleotides to about 60 nucleotides, and preferably from about 12 to about 30 nucleotides. In one embodiment, the hook sequence and the hook complement sequence are each about 15 nucleotides.

While in the broadest sense the hook sequence or the hook complement sequence may have any random sequence, the following parameters are preferably employed to identify an appropriate sequence. First, the oligonucleotide sequence should be a sequence which is rare with respect to sequences present in the organism from which nucleic acid to be targeted is derived. Preferably, the hook sequence is not a naturally-occurring sequence in the organism of the cell to which the targeted nucleic acid is to be delivered. Rarity of a particular sequence, and therefore its suitability for use in a targeting molecule, can be identified using any convenient sequence similarity comparison tool, such as BLAST searches, and the like. In addition, the sequence should possess minimal self structure, e.g. it should not form hairpin loops or other secondary structures. Finally, as indicated above, the hook complement sequence preferably possesses a sufficiently high melting temperature when hybridized to its complementary sequence, e.g. the hook sequence. By sufficiently high melting temperature is meant a melting temperature wherein at least about 10%, more preferably at least about 25%, and more preferably still at least about 50% of the targeted nucleic acid remains bound to the targeting molecule in order to reach, bind and, preferably, be internalized by the target cell. In some embodiments, a sufficiently high melting temperature equals or exceeds at least about 37° C. Optimizing the melting temperature of the hook sequence for a particular application is readily achieved by the skilled artisan using conventional methods and knowledge in the art. The invention should not be construed as limited by the melting temperature of the hook sequence. Hook complement sequences and hook sequences suitable for use in the present invention can be designed manually or with the assistance of a computing means, in which an algorithm is employed that is capable of identifying sequences that satisfy the above-described parameters. Such algorithms are available in the art (see, for instance, Chavali et al., 2005, Bioinformatics 21(20):3918-3925).

The oligonucleotide of the targeting molecule may be DNA or RNA. In one embodiment, the oligonucleotide is DNA. In another embodiment, the oligonucleotide is RNA. The oligonucleotide may comprise modifications, as described elsewhere herein, such as phosphate backbone modification, nucleotide sugar modification, nucleotide base modification, and non-nucleotide modification or any combination of these modifications. The oligonucleotide component of the targeting molecule may be prepared by any conventional means typically used to prepare nucleic acids in large quantity. For example, DNAs and RNAs may be chemically synthesized using commercially available reagents and synthesizers by methods that are well-known in the art (see, e.g., Gait, 1985, Oligonucleotide Synthesis: A Practical Approach (IRL Press, Oxford, England)). Alternatively, DNAs and RNAs are enzymatically synthesize in vitro or in vivo using conventional methods of molecular biology. See, for instance, Sambrook et al. (2001, Molecular Cloning: A Laboratory Manual, Cold Spring Harbor Laboratory Press, Cold Spring Harbor, N.Y.), Ausubel et al. (eds., 2005, Current Protocols in Molecular Biology, John Wiley & Sons, New York), and Gerhardt et al. (eds., 1994, Methods for General and Molecular Bacteriology, American Society for Microbiology, Washington, D.C.).

The receptor binding moiety of the targeting molecule may be any molecule that binds to a receptor and induces receptor-mediated endocytosis. Non-limiting examples of a receptor that undergoes endocytosis upon binding to a cognate receptor binding moiety include folate receptor; integrins; epidermal growth factor (EGR) receptor; transferrin receptor; low density lipoprotein (LDL) receptor; insulin and other protein hormone receptors; galactosamine receptor; galactose receptor; biotin receptor; platelet-derived growth factor receptor; thyrotrypsin releasing hormone (TRH) receptor; nerve-growth factor (NGF) receptor; and any of the receptors for various specific viral factors, e.g., a specific viral antigen of the HIV virus specific to the T4-receptor typical of T4 lymphocytes but which can also be found on other cells (see Maddon et al., 1986, Cell 47:333); Ct₂-macroglobulin receptor; thiodothyronine receptor; thrombine receptor; arachidonic acid receptor; transforming growth factor-α (TGF-α) receptor; receptors for the various heregulins (HRGs); alpha fetoprotein (AFP) receptor, and the like. In preferred embodiments, the cognate receptor for the receptor binding moiety is a folate receptor or an integrin. Integrins are receptors for adhesion proteins and extracellular matrix components, such as fibronectin, vitronectin, collagen and laminin. The integrin-binding activity of adhesion proteins can be reproduced by short synthetic peptides containing the RGD sequence (Ruoslahti, 1996, Annu Rev Cell Dev Biol. 12:697-715). Accordingly, in preferred embodiments, the receptor binding moiety of the targeting molecule is folate or an RDG-containing peptide or peptidomimetic thereof.

The term “folate” as used herein, refers to folic acid and analogs and derivatives thereof, which can bind and induce folate receptor mediated endocytosis. Non-limiting examples of analogs and derivatives include dihydrofloates, tetrahydrofolates, tetrahydrorpterins, folinic acid, pteropolyglutamic acid, deaza analogs, dideaza analogs, and pteroic acid derivatives. The terms “deaza analogs” and “dideaza analogs’ refer to the art recognized analogs having a carbon atom substituted for one or two nitrogen atoms in the naturally occurring folic acid structure. For example, deaza analogs include the 1-deaza, 3-deaza, 5-deaza, 8-deaza, and 10-deaza analogs. Dideaza analogs include, for example, 1,5 dideaza, 5,10-dideaza, 8,10-dideaza, and 5,8-dideaza analogs. Other folates useful as receptor binding moieties are the folate receptor-binding analogs aminopterin, amethopterin (methotrexate), N¹⁰-methylfolate, 2-deamino-hydroxyfolate, deaza analogs such as 1-deazamethopterin or 3-deazamethopterin, and 3′5′-dichloro-4-amino-4-deoxy-N¹⁰-methylpteroylglutamic acid (dichloromethotrexate). Other suitable ligands capable of binding to folate receptors to initiate receptor-mediated endocytotic transport of the complex include anti-idiotypic antibodies to the folate receptor. Folate mimetics are disclosed in U.S. Patent Publication No. 20050227985.

RGD-containing peptides, derivatives and peptidomimetics are known in the art. See, for instance, U.S. Patent Publication Nos. 20020103130 and 20050069494; Koivunen et al., 1993, J Biol. Chem. 268(27):20205-10; Koivunen et al. 1995, Biotechnology 13(3):265-170; Ruoslahti, 1996, Annu Rev Cell Dev Biol. 12:679-715; Holig et al., 2004, Protein Eng Des Sel. 17:433-441; Temming et al., 2005, Drug Resist Updat. 8(6):381-402. Epub 2005 Nov. 23 and Mitra et al., 2005, J. Controlled Release 102: 19. Cyclic RGD-containing peptides useful in the invention are disclosed, for instance, in U.S. Pat. Nos. 5,866,540 and 6,001,961. U.S. Pat. No. 6,627,769 discloses non-peptidic RGD mimetics. Any such peptide, derivative or peptidomimetic is useful in the method of the invention. In one embodiment, the RGD-containing peptide is ACDCRGDCFC (amino acids 1-10 of SEQ ID NO. 6) or a derivative thereof.

An RGD-containing peptide may be prepared by chemical or biological means. Biological methods include, without limitation, expression of a nucleic acid encoding an RGD-containing peptide in a host cell or in an in vitro translation system, using methods well known to the skilled artisan.

An RGD-containing peptide may be chemically synthesized by Merrifield-type solid phase peptide synthesis. This method may be routinely performed to yield peptides up to about 60-70 residues in length, and may, in some cases, be utilized to make peptides up to about 100 amino acids long. Larger peptides may also be generated synthetically via fragment condensation or native chemical ligation (Dawson et al., 2000, Ann. Rev. Biochem. 69:923-960). An advantage to the utilization of a synthetic peptide route is the ability to produce large amounts of peptides, even those that rarely occur naturally, with relatively high purities, i.e., purities sufficient for research, diagnostic or therapeutic purposes.

Solid phase peptide synthesis is described by Stewart et al. in Solid Phase Peptide Synthesis, 2nd Edition, 1984, Pierce Chemical Company, Rockford, Ill.; and Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and coupling thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group, such as formation into a carbodiimide, a symmetric acid anhydride, or an “active ester” group, such as hydroxybenzotriazole or pentafluorophenyl esters.

Examples of solid phase peptide synthesis methods include the BOC method, which utilizes tert-butyloxcarbonyl as the α-amino protecting group, and the FMOC method, which utilizes 9-fluorenylmethyloxcarbonyl to protect the α-amino of the amino acid residues. Both methods are well-known by those of skill in the art.

Incorporation of N- and/or C-blocking groups may also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin, so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB (divinylbenzene), resin, which upon hydrofluoric acid (HF) treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by trifluoroacetic acid (TFA) in dicholoromethane. Esterification of the suitably activated carboxyl function, e.g. with dicyclohexylcarbodiimide (DCC), can then proceed by addition of the desired alcohol, followed by de-protection and isolation of the esterified peptide product.

Incorporation of N-terminal blocking groups may be achieved while the synthesized peptide is still attached to the resin, for instance by treatment with a suitable anhydride and nitrile. To incorporate an acetyl blocking group at the N-terminus, for instance, the resin-coupled peptide can be treated with 20% acetic anhydride in acetonitrile. The N-blocked peptide product may then be cleaved from the resin, de-protected and subsequently isolated.

Prior to its use in a targeting molecule of the invention, an RGD-containing peptide is purified to remove contaminants. In this regard, it will be appreciated that the peptide will be purified so as to meet the standards set out by the appropriate regulatory agencies. Any one of a number of a conventional purification procedures may be used to attain the required level of purity including, for example, reversed-phase high-pressure liquid chromatography (HPLC) using an alkylated silica column such as C₄-, C₈- or C₁₈-silica. A gradient mobile phase of increasing organic content is generally used to achieve purification, for example, acetonitrile in an aqueous buffer, usually containing a small amount of trifluoroacetic acid. Ion-exchange chromatography can be also used to separate polypeptides based on their charge. Affinity chromatography is also useful in purification procedures.

To prepare a targeting molecule, the receptor-binding moiety is conjugated to the oligonucleotide component by methods known in the art. The art is replete with conjugation chemistries useful for such conjugation. For instance, folate can be conjugated with the oligonucleotide component of a targeting molecule using art-recognized covalent coupling techniques disclosed, for instance, in U.S. Pat. Nos. 5,416,016, 6,335,435, 6,528,631, 6,861,514 and 6,919,439, incorporated herein by reference in their entirety. Other conjugation chemistries are disclosed in U.S. Patent Publication No. 20040249178, incorporated herein by reference in its entirety. A single receptor binding moiety may be attached to the oligonucleotide or a plurality of binding moieties may be attached. A receptor binding moiety or a linker useful for conjugation to a receptor binding moiety can be introduced at one or more locations in an oligonucleotide during solid phase synthesis and/or in post-synthetic coupling approaches. See, for instance, U.S. Patent Publication No. 2004/0192626, incorporated herein by reference in its entirety and references cited therein. Conjugation chemistries for linking a peptide to an oligonucleotide to prepare a targeting molecule are also known in the art. Non-limiting examples include p-hydroxy-benzoic acid linkers (Chang-Po et al., 2002, Bioconjugate Chem. 13(3):525-529); native ligation (Stetsenko et al., 2000, J Org. Chem. 65:4900-4908): disulfide bridge conjugates (Oehlke et al., 2002, Eur J. Biochem. 269:4025-4032 and Rogers et al., 2004, Nuc Acids Res. 32(22) 6595-6604); maleimide linkers (Zhu et al., 1993, Antisense Res Dev. 3:265-275; thioester linkers (Ede et al., 1994, Bioconjug Chem. 5:373-378); Diels-Alder cycloaddition (Marchán et al., 2006. Nuc Acids Res. 34(3): e24, 2006 Feb. 14 Epub); U.S. Pat. No. 6,656,730 and the like. For reviews of peptide-oligonucleotide conjugation chemistries, see Tung et al., 2000, Bioconjugate Chem. 11:605-618; Zatsepin et al., 2005, Curr Pharm Des. 11(28):3639-3654; and Juliano, 2005, Curr Opin Mol. Ther. 7(2):132-136. Therefore, the skilled artisan has sufficient guidance for preparing a targeting molecule of the invention comprising, for instance, an RGD-containing peptide conjugated to an oligonucleotide.

Any nucleic acid of interest may be targeted using the method of the invention. It is not intended that the present invention be limited by the nature of the nucleic acid employed. The targeted nucleic acid may be native, naturally-occurring nucleic acid or synthesized nucleic acid. The nucleic acid may be from a viral, bacterial, animal or plant source. The nucleic acid may be DNA or RNA and may exist in a double-stranded, single-stranded or partially double-stranded form. Nucleic acids may also be in triplex or partial triplex form. Furthermore, the nucleic acid may be found as part of a virus or other macromolecule. See, e.g., Fasbender et al., 1996, J Biol. Chem. 272:6479-89. Therefore, nucleic acids that can be targeted in the method of the present invention include, by way of example and not limitation, oligonucleotides and polynucleotides such as any single-stranded or double-stranded nucleic acid, antisense DNAs and/or RNAs; ribozymes; DNA for gene therapy; viral fragments including viral DNA and/or RNA; DNA and/or RNA chimeras; mRNA; short interfering nucleic acid (siNA); short interfering RNA (siRNA); microRNA (miRNA); short hairpin RNA (shRNA); antagomirs; plasmids; cosmids; genomic DNA; cDNA; gene fragments; various structural forms of DNA including single-stranded DNA, doublestranded DNA, supercoiled DNA and/or triple-helical DNA; Z-DNA; and the like. Generally, there are no size limitations on the nucleic acid that is targeted in accordance with the method of the invention. Optionally, for large nucleic acids, a nucleic acid condensation reagent may be used to condense the nucleic acid bound to a targeting molecule. Condensation reagents are known in the art and commercially available. In preferred embodiments, the nucleic acid targeted is an siRNA, an miRNA, an antagomir or an shRNA. In even more preferred embodiments, the nucleic acid is an siRNA. An antagomir is a chemically-engineered oligonucleotide that can specifically silence target miRNA expression (Krutzfeldt et al., 2005, Nature 438:685-689). miRNAs are closely related to siRNAs. These miRNAs represent an abundant and important class of ˜22-nt noncoding RNA species in both animals and plants. miRNAs are involved in many processes, including regulation of gene expression during development and cell growth control (Ambros, 2004, Nature Rev Genet. 5:396-400). While they are not generated from perfect duplex RNA precursors and do not, in general, act by perfectly matching their targets through complementary base-pairing, miRNAs nevertheless appear to function through the same underlying cellular RNAi machinery as siRNAs. There are at least 300 miRNAs found in humans (reviewed in Bartel, 2004, Cell 116:281-297 and Kim, 2005, Nat Rev Mol Cell Biol. Epub 2005 Apr. 15). Binding of miRNAs to target mRNAs generally results in translational repression (Ambros, 2004, Nature 431:350-355). Unique miRNA expression profiles have been seen in a variety of cancer cells, suggesting that miRNAs can contribute to cancer development and progression (Esquela-Kerscher et al., 2006, Nat Rev Cancer 6:259-269). Therefore inhibition of miRNAs can be a potential therapeutic for cancer gene therapy.

The term “short interfering nucleic acid,” “siNA,” “short interfering RNA,” “siRNA,” “short interfering nucleic acid molecule,” “short interfering oligonucleotide molecule,” or “chemically-modified short interfering nucleic acid molecule” as used herein refers to any nucleic acid molecule capable of inhibiting or down regulating gene expression or viral replication, for example by mediating RNA interference (RNAi) or gene silencing in a sequence-specific manner. See for example: Zamore et al., 2000, Cell 101: 25-33; Bass, 2001, Nature 411: 428-429; Elbashir et al., 2001, Nature 411: 494-498; and Kreutzer et al., International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914; Allshire, 2002, Science 297:1818-1819; Volpe et al., 2002, Science 297: 1833-1837; Jenuwein, 2002, Science 297: 2215-2218; Hall et al., 2002, Science 297: 2232-2237; Hutvagner and Zamore, 2002, Science 297: 2056-60; McManus et al., 2002, RNA 8: 842-850; Reinhart et al., 2002, Gene & Dev. 16: 1616-1626; and Reinhart et al., 2002, Science 297:1831).

An siNA or siRNA can be a double-stranded polynucleotide molecule comprising self-complementary sense and antisense regions, wherein the antisense region comprises a nucleotide sequence that is complementary to nucleotide sequence in a target nucleic acid molecule or a portion thereof and the sense region has a nucleotide sequence corresponding to the target nucleic acid sequence or a portion thereof. The siNA or siRNA can be assembled from two separate oligonucleotides, where one strand is the sense strand and the other is the antisense strand, wherein the antisense and sense strands are self-complementary (i.e. each strand comprises nucleotide sequence that is complementary to nucleotide sequence in the other strand; such as where the antisense strand and sense strand form a duplex or double stranded structure, for example wherein the double stranded region is about 19 base pairs); the antisense strand comprises nucleotide sequence that is complementary to a nucleotide sequence in a target nucleic acid molecule or a portion thereof and the sense strand comprises a nucleotide sequence corresponding to the target nucleic acid sequence or a portion thereof. A least one of the two oligonucleotides further comprises a hook sequence to enable base-pairing to the targeting oligonucleotide. The sense oligonucleotide can be connected to the antisense oligonucleotide via a linker molecule, such as a polynucleotide linker or a non-nucleotide linker. Alternatively, the siNA is assembled from a single oligonucleotide, comprising a hook sequence, where the self-complementary sense and antisense regions of the siNA are linked by means of a nucleic acid based or non-nucleic acid-based linker(s). The siNA or siRNA can be a polynucleotide with a duplex, asymmetric duplex, hairpin or asymmetric hairpin secondary structure, having self-complementary sense and antisense regions, wherein the antisense region comprises nucleotide sequence that is complementary to a nucleotide sequence in a separate target nucleic acid molecule or a portion thereof and the sense region having a nucleotide sequence corresponding to the target nucleic acid sequence or a portion thereof.

Chimeric or gapmer siNAs and siRNAs such as those disclosed in U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, incorporated herein by reference in their entirety, are also encompassed in the invention.

Modified nucleic acids may be used in the method of the invention. Non-limiting examples of such chemical modifications independently include without limitation phosphate backbone modification (e.g. phosphorothioate internucleotide linkages), nucleotide sugar modification (e.g., 2′-O-methyl nucleotides, 2′-O-allyl nucleotides, 2′-deoxy-2′-fluoro nucleotides, 2′-deoxyribonucleotides), nucleotide base modification (e.g., “universal base” containing nucleotides, 5-C-methyl nucleotides), and non-nucleotide modification (e.g., abasic nucleotides, inverted deoxyabasic residue) or a combination of these modifications. In addition, oligonucleotides having morpholino backbone structures (U.S. Pat. No. 5,034,506) or polyamide backbone structures (Nielsen et al., 1991, Science 254: 1497) may also be used. These and other chemical modifications can preserve biological activity of the targeted nucleic acid in vivo while at the same time, dramatically increasing the serum stability, potency, duration of effect and/or specificity of these compounds. Nucleic acids containing modified internucleoside linkages may also be synthesized using reagents and methods that are well known in the art. For example, methods for synthesizing nucleic acids containing phosphonate phosphorothioate, phosphorodithioate, phosphoramidate methoxyethyl phosphoramidate, formacetal, thioformacetal, diisopropylsilyl, acetamidate, carbamate, dimethylene-sulfide (—CH₂—S—CH₂), dimethylene-sulfoxide (—CH₂—SO—CH₂), dimethylene-sulfone (—CH₂—SO₂—CH₂), 2′-O-alkyl, and 2′-deoxy-2′-fluoro phosphorothioate internucleoside linkages are well known in the art (see Uhlmann et al., 1990, Chem. Rev. 90:543-584; Schneider et al., 1990, Tetrahedron Lett. 31:335 and references cited therein).

A nucleic acid molecule useful in the invention may have one or more modified pyrimidine and/or purine nucleotides. In one embodiment, the pyrimidine nucleotides in the sense region are 2′-O-methylpyrimidine nucleotides or 2′-deoxy-2′-fluoro pyrimidine nucleotides and the purine nucleotides present in the sense region are 2′-deoxy purine nucleotides. In another embodiment, the pyrimidine nucleotides in the sense region are 2′-deoxy-2′-fluoro pyrimidine nucleotides and the purine nucleotides present in the sense region are 2′-O-methyl purine nucleotides. In another embodiment, the pyrimidine nucleotides in the sense region are 2′-deoxy-2′-fluoro pyrimidine nucleotides and the purine nucleotides present in the sense region are 2′-deoxy purine nucleotides. In one embodiment, the pyrimidine nucleotides in the antisense region are 2′-deoxy-2′-fluoro pyrimidine nucleotides and the purine nucleotides present in the antisense region are 2′-O-methyl or 2′-deoxy purine nucleotides. In another embodiment of any of the above-described nucleic acid molecules, any nucleotides present in a non-complementary region of the sense strand (e.g. overhang region) are 2′-deoxy nucleotides.

The sense strand of a dsRNA molecule for RNAi can be modified in order to inactivate the sense strand and prevent formation of an active RISC, thereby potentially reducing off-target effects. This can be accomplished by a modification which prevents 5′-phosphorylation of the sense strand, e.g., by modification with a 5′-O-methyl ribonucleotide (see Nykanen et al., 2001, Cell 107: 309-321). Other modifications which prevent phosphorylation can also be used, e.g., simply substituting the 5′-OH by H rather than O-Me. Alternatively, a large bulky group may be added to the 5′-phosphate turning it into a phosphodiester linkage.

Nucleic acids useful in the invention may comprise a chemical modification called a cap structure incorporated at either terminus of the oligonucleotide (see, for example, U.S. Pat. No. 5,998,203, and WO 03/70918 incorporated by reference herein). These terminal modifications protect the nucleic acid molecule from exonuclease degradation, and can help in delivery and/or localization within a cell. The cap can be present at the 5′-terminus (5′-cap) or at the 3′-terminal (3′-cap) or can be present on both termini. Non-limiting examples of the 5′-cap include, but are not limited to, glyceryl, inverted deoxy abasic residue (moiety); 4′,5′-methylene nucleotide; I-(beta-D-erythrofuranosyl) nucleotide, 4′-thio nucleotide; carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety.

Non-limiting examples of the 3′-cap include, but are not limited to, glyceryl, inverted deoxy abasic residue (moiety), 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate; 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage et al., 1993, Tetrahedron 49: 1925; incorporated by reference herein).

The examples of oligonucleotide modifications described herein are not exhaustive and it is understood that the invention includes additional modifications of the oligonucleotides of the invention which modifications serve to enhance the therapeutic or other properties of the oligonucleotides without appreciable alteration of the basic sequence of the oligonucleotide.

Double-stranded nucleic acids can comprise mismatches (e.g., 1, 2, 3 or 4 mismatches), bulges, loops, or wobble base pairs, for example, to modulate or regulate the ability of the nucleic acid to mediate inhibition of gene expression. Mismatches, bulges, loops, or wobble base pairs may be introduced into the duplex molecules to the extent such mismatches, bulges, loops, or wobble base pairs do not significantly impair the ability of the duplex nucleic acids to mediate inhibition of target gene expression. Such mismatches, bulges, loops, or wobble base pairs may be present in regions of the duplex that do not significantly impair the ability of such nucleic acids to mediate inhibition of gene expression, for example, mismatches may be present at the terminal regions of the duplex or at one or positions in the internal regions of the duplex. Similarly, the wobble base pairs may, for example, be at the terminal base paired region(s) of the duplex or in the internal regions or in the regions where palindromic sequences are present withing the duplex oligonucleotide.

The nucleic acids may be prepared by any conventional means typically used to prepare nucleic acids in large quantity. For example, DNAs and RNAs may be chemically synthesized using commercially available reagents and synthesizers by methods that are well-known in the art (see, e.g., Gait, 1985, Oligonucleotide Synthesis: A Practical Approach (IRL Press, Oxford, England)). RNAs may be produced in high yield via in vitro transcription using plasmids such as SP65 (Promega Corporation, Madison, Wis.). The nucleic acids may be purified by any suitable means, as are well known in the art. For example, the nucleic acids can be purified by reverse phase or ion exchange HPLC, size exclusion chromatography or gel electrophoresis. Of course, the skilled artisan will recognize that the method of purification will depend in part on the size of the nucleic acid to be purified.

In a single stranded nucleic acid to be targeted according to the method of the invention, the strand comprises a hook sequence. In a double stranded nucleic acid, at least one strand comprises a hook sequence. In one embodiment, the sense strand of a double stranded nucleic acid comprises a hook sequence. In another embodiment, the antisense strand of a double stranded nucleic acid comprises a hook sequence. In yet another embodiment, both strands of a double stranded nucleic acid to be targeted comprise a hook sequence. The hook sequence may be located anywhere within a nucleic acid to be targeted. In preferred embodiments, the hook sequence flanks the nucleic acid of interest, such as the antisense or sense sequence of an siRNA. More preferably, the hook sequence is located at or in close proximity to the 5′ or the 3′ of a nucleic acid molecule.

The hook sequence is added to the nucleic acid to be targeted using any method known in the art. The hook sequence may be synthesized as part of the nucleic acid. For instance, the hook sequence may be incorporated into a primer used in a polymerase chain reaction (PCR) to amplify a nucleic acid of interest to be targeted in the method of the invention. Alternatively, an expression cassette for in vitro or in vivo transcription can be recombinantly engineered to comprise the hook sequence flanking the nucleic acid to be targeted using methods known to the skilled artisan. The hook sequence can also be synthesized separately from the nucleic acid to be targeted and be covalently attached to the nucleic acid by means known in the art. Standard nucleic acid coupling chemistries include, for instance, standard direct enzymatic ligation using DNA or RNA ligases, or DNA-bridged RNA-RNA ligation (Moore et al., 1992, Science 256:992-997). Alternatively, two nucleic acid fragments with linker elements at their ends can be joined together via non-enzymatic reactions. See, for instance, Xu et al., 1997, Tetrahedron Lett. 38:5595-5598 and U.S. Pat. No. 5,476,930. Bioconjugation services are commercially available, for instance, from SoluLink, Inc. (San Diego, Calif.).

To prepare a targeted nucleic acid, a targeting molecule is mixed with the nucleic acid to be targeted. The oligonucleotide component of the targeting molecule is allowed to hybridize to the hook sequence of the nucleic acid. The skilled artisan is familiar with conditions for facilitating hybridization of substantially complementary or 100% complementary sequences, such as the hook sequence and the hook complement sequence. Hybridization is influenced by temperature, ionic strength of the annealing medium, the incubation period, the length of the sequences to hybridize, the G-C content, and the extent of the homology of the sequences to be hybridized. Generally, a targeting molecule is mixed with a nucleic acid to be targeted in solution, and the mixture is incubated at a temperature equal to or in excess of the predicted melting temperature of the hook sequence and its complement. Preferably the incubation temperature is in excess of the predicted melting temperature. If the nucleic acid to be targeted is double stranded and the nucleic acid is hybridized prior to the addition of the targeting molecule, the incubation temperature is optionally less than the predicted melting temperature of the double-stranded nucleic acid, to minimize the melting of the already-hybridized portion. In another embodiment, the targeting molecule and the two complementary strands of a duplex nucleic acid to be targeted are mixed together and allowed to hybridize concurrently. In this embodiment, the incubation temperature is preferably in excess of the higher of the two predicted melting temperatures (hook sequence and hook complement sequence; two complementary strands of the duplex nucleic acid). The solution is maintained at an elevated temperature for a suitable period of time and then cooled, passively or actively. The targeting molecule is added to the nucleic acid to be targeted in at least a 1:1 molar ratio with respect to the number of hook sequences. Preferably, the targeting oligonucleotide is added in molar excess to maximize the preparation of targeted nucleic acids.

Prior to its use in a method of delivering the nucleic acid to a cell, the targeted nucleic acid may be purified away from unhybridized molecules using standard methods in the art, including, but not limited to, affinity chromatography, size exclusion chromatography, ion-exchange chromatography, high pressure liquid chromatography and the like. The targeted nucleic acid is then administered so as to contact a cell which expresses the cognate receptor for the receptor-binding moiety of the targeting oligonucleotide molecule. Without wishing to be bound by theory, the receptor-binding moiety of the targeted nucleic acid binds to its cognate receptor and the targeted nucleic acid is internalized by receptor-mediated endocytosis, thereby delivering the targeted nucleic acid. However, the invention should not be construed as being limited to this mechanism of internalization.

The method of the invention can be used in any application requiring or involving the cellular targeting of a nucleic acid. The methods may be used for diagnostic applications, research applications and therapeutic applications. The method may be in vitro, ex vivo or in vivo. For example, the method of the invention may be used to introduce a nucleic acid into tissue or cells ex vivo that are subsequently transplanted into a subject for therapeutic effect. The cells and/or tissue can be derived from an organism or subject that later receives the explant, or can be derived from another organism or subject prior to transplantation. The nucleic acid targeted to the cell or tissue can be used to modulate the expression of one or more genes in the cells or tissue, such that the cells or tissue obtain a desired phenotype or are able to perform a function when transplanted in vivo. Non-limiting examples of ex vivo applications include use in organ/tissue transplant, tissue grafting, or treatment of pulmonary disease (e.g., restenosis) or prevent neointimal hyperplasia and atherosclerosis in vein grafts. Such ex vivo applications may also be used to treat conditions associated with coronary and peripheral bypass graft failure, for example, such methods can be used in conjunction with peripheral vascular bypass graft surgery and coronary artery bypass graft surgery. Additional applications include transplants to treat CNS lesions or injury, including use in treatment of neurodegenerative conditions such as Alzheimer's disease, Parkinson's Disease, epilepsy, dementia, Huntington's disease, or amyotrophic lateral sclerosis (ALS).

The method of the invention provides a novel therapeutic approach to a broad spectrum of diseases and conditions, including cancer or cancerous disease, infectious disease, ocular disease, cardiovascular disease, neurological disease, prion disease, inflammatory disease, autoimmune disease, pulmonary disease, renal disease, liver disease, mitochondrial disease, endocrine disease, reproduction related diseases and conditions, and any other indications that can respond to the level of an expressed gene product or a foreign nucleic acid, such as viral, fungal or bacterial genome, in a cell or organism.

In one embodiment, the nucleic acid encodes a therapeutic molecule. The therapeutic molecule may be any therapeutic molecule that can be encoded in a polynucleotide or whose production in vivo can be modulated by a molecule encoded in a polynucleotide. Non-limiting examples of types of therapeutic molecules that can be encoded in an expression cassette in the instant invention include, but are not limited to, polypeptide enzymes, cytokines, hormones, antibodies, such as intrabodies or scFvs, a suicide gene, such as HSV-TK, a molecule that inhibits vascularization, a molecule that increases vascularization, tumor suppressors, such as p53 and p21, pro-apoptotic molecules, such as TRAIL, transcription factors, receptors, ligands, immunogenic molecules, anti-proliferative molecules, agonists, antagonists, anti-inflammatory molecules, antibiotics, antidepressants, prodrugs, anti-hypertensives, anti-oxidants, and the like.

In another embodiment, the present invention features methods to modulate gene expression, for example, of genes involved in the progression and/or maintenance of cancer or in a viral infection. For example, in one embodiment, the invention features the use of one or more of the targeted nucleic acids of the invention independently or in combination to inhibit the expression of the gene(s) encoding proteins associated with cancerous conditions, for example breast cancer, lung cancer, colorectal cancer, brain cancer, esophageal cancer, stomach cancer, bladder cancer, pancreatic cancer, cervical cancer, head and neck cancer, ovarian cancer, melanoma, lymphoma, glioma, or multidrug resistant cancer associated genes. In another embodiment, the invention features the use of one or more targeted nucleic acids independently or in combination to inhibit the expression of the gene(s) encoding viral proteins, for example human immunodeficiency virus (HIV), hepatitis B virus (HBV), hepatitis C virus (HCV), cytomegalovirus (CMV), respiratory syncytial virus (RSV), herpes simplex virus (HSV), poliovirus, influenza, rhinovirus, West Nile virus, Ebola virus, foot and mouth virus, severe acute respiratory syndrome-associated coronavirus (SARS-CoV) and papilloma virus associated genes.

The art is replete with exemplary molecules and associated diseases or disorders where a patient may benefit from the expression or inhibition of expression of one or more molecules. For instance, an assessment of expression changes in gene families in a variety of human cancers has been pursued U.S. Pat. Appl. Pub. No. 20060168670. In addition, tissue-specific expression levels have been mapped for thousands of genes through expression profiling. Alon et al., 1999, Proc. Natl. Acad. Sci. USA 96:6745-50; Iyer et al., 1999, Science 283: 83-87; Khan et al., 1998, Cancer Res. 58: 5009-13; Lee et al., 1999, Science 285:1390-93; Wang et al., 1999, Gene 229:101-08; and Whitney et al., 1999, Ann. Neurol. 46:42. Thus, the skilled artisan is able to select molecules useful in the practice of the present invention without undue experimentation.

In one embodiment, the nucleic acid is an siRNA and the oligonucleotide of the targeting molecule is DNA. In one embodiment, the nucleic acid is an siRNA that targets a gene associated with a cancer. In one embodiment, the siRNA targets a gene that contributes to the development of chemoresistance (e.g. MyD88). In another embodiment, the siRNA targets a gene that decreases or represses apoptosis (e.g. XIAP and survivin).

Folate receptors, while expressed at negligible levels on the surface of most normal tissue cells, are over-expressed on human nasopharyngeal carcinoma and human epithelium ovarian carinomas. Thus, targeting nucleic acids via the folate receptor in the method of the invention is useful for delivering nucleic acids to such tissues. Hepatocytes express galatose and galatosamine receptors, thus, nucleic acids targeted to these receptors are useful for treating liver diseases, including, but not limited to HBV or HCV infection. T-lymphocytes express CD4 receptors, therefore, nucleic acids targeted to CD4 receptors can be useful in diseases or disorders of T lymphocytes, such as HIV. The αᵥβ₃/αᵥβ₅ integrins are often significantly over-expressed on endothelial cells of neovasculature and on cancer cells, including lung cancer, breast cancer and melanoma. Thus, the method of the invention can be used to target nucleic acids to angiogenic endothelial cells using RGD-containing peptides, derivatives or peptidomimetics thereof.

The invention also encompasses the use of pharmaceutical compositions of a targeted nucleic acid to practice the methods of the invention, the compositions comprising a targeted nucleic acid of the invention and a pharmaceutically-acceptable carrier. As used herein, the term “pharmaceutically-acceptable carrier” means a chemical composition with which a targeted nucleic acid in accordance with the invention may be combined and which, following the combination, can be used to administer the targeted nucleic acid to a mammal.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

The pharmaceutical compositions useful for practicing the invention may be administered to deliver a dose of between 1 ng/kg/day and 100 mg/kg/day. In one embodiment, the invention envisions administration of a dose which results in a concentration of the compound of the present invention between 1 μM and 10 μM in a mammal.

Typically, dosages which may be administered in a method of the invention to an animal, preferably a human, range in amount from 0.5 μg to about 50 mg per kilogram of body weight of the animal. While the precise dosage administered will vary depending upon any number of factors, including but not limited to, the type of animal and type of disease state being treated, the age of the animal and the route of administration. Preferably, the dosage of the targeted nucleic acid will vary from about 1 μg to about 10 mg per kilogram of body weight of the animal. More preferably, the dosage will vary from about 3 μg to about 1 mg per kilogram of body weight of the animal.

The targeted nucleic acid may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit. The active ingredient may be present in the pharmaceutical composition in the form of a physiologically acceptable ester or salt, such as in combination with a physiologically acceptable cation or anion, as is well known in the art.

As used herein, the term “physiologically acceptable” ester or salt means an ester or salt form of the active ingredient which is compatible with any other ingredients of the pharmaceutical composition, which is not deleterious to the subject to which the composition is to be administered.

For oligonucleotides and nucleic acids, preferred examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

Pharmaceutical compositions that are useful in the methods of the invention may be prepared, packaged, or sold in formulations suitable for parenteral, ophthalmic, topical, pulmonary, buccal, intranasal, oral, rectal, vaginal, or another route of administration. In addition to the targeted nucleic acid and pharmaceutically-acceptable carrier, the pharmaceutical compositions may contain other ingredients known to enhance and facilitate drug administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intravenous, intraocular, intravitreal, subcutaneous, intraperitoneal, intramuscular, intrasternal injection, intratumoral, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, in microbubbles for ultrasound-released delivery, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may further comprise buffering agents, salts, or one or more other of the additional ingredients described herein. Other ophthalmically-administrable formulations which are useful include those which comprise the active ingredient in microcrystalline form or in a liposomal preparation, provided the receptor-binding moiety of the targeting molecule is accessible to target delivery to the cognate receptor.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, Pa.), which is incorporated herein by reference.

The invention also provides kits useful for carrying out a method of the invention. The kit comprises a targeting molecule and an instructional material for using the targeting molecule to target a nucleic acid. In one embodiment, the receptor-binding moiety is one of folate or an RGD-containing peptide, derivative or peptidomimetics thereof. In one embodiment, the kit further comprises an oligonucleotide, comprising a hook sequence corresponding to the hook complement sequence in the targeting oligonucleotide, and instructions for attaching the oligonucleotide to a nucleic acid of interest. Optionally, the kit further comprises an applicator. The instructional material in each kit simply embody the disclosure provided herein.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Experimental Example 1

### Cellular Delivery of Folate-Conjugated Oligonucleotides

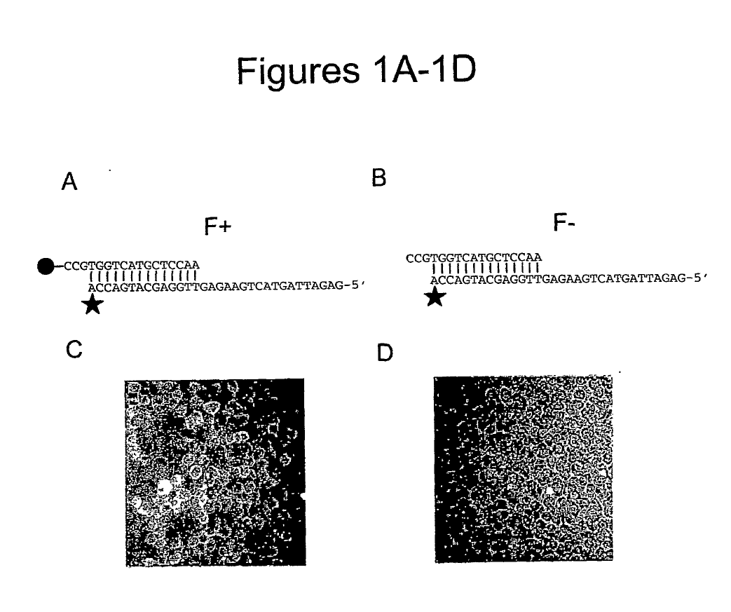

To test the feasibility of a folate-conjugated oligonucleotide (F+ODN) to deliver a nucleic acid cargo to folate receptor (FR)-expressing cells via base-paired interaction, two oligonucleotides were designed that can form a partial duplex with each other (FIG. 1A). To prepare the targeting molecule, folate was conjugated to N-hydroxysulfosuccinimide (NHS) as described in Aronov et al., 2003, Bioconjug Chem. 14:563-574. The free amino group at the 5′-end of the tethering oligonucleotide (a 17-mer; SEQ ID NO. 1) was then covalently conjugated to the folate-NHS (FIG. 1A; carried out in the laboratory of Dr. Phil Low, Purdue University). The nucleic acid cargo to be delivered was a 32-mer oligonucleotide (SEQ ID NO. 2) with its 3′-end labeled by a fluorescein tag (Yale University Keck facility). The sequences of both oligonucleotides were randomly chosen, other than assuring a complementary sequence to permit basepairing of one oligonucleotide to the other. As a negative control, the same oligonucleotide (SEQ ID NO. 1) but lacking conjugated folic acid (FIG. 1B) was used in a parallel experiment, in a duplex (F−) with SEQ ID NO. 2 with its 3′-end labeled by a fluorescein tag.

The F+ oligonucleotide (SEQ ID NO. 1) and the unconjugated oligonucleotide (SEQ ID NO. 1) were each mixed separately with the fluorescein-labeled cargo strand (SEQ ID NO. 2) in a 1:1 molar ratio, denatured by heating at 90° C. for 3 minutes, and annealed by slowly cooling down to room temperature for 1 hour. The annealed duplexes (F+ and F−) were then separately added to KB cells, an oral epidermoid carcinoma cell line which overexpresses the folate receptor, at a final concentration of 100 nM in folate-free RPMI 1640 culture medium in the absence of serum. The cells were then incubated at room temperature (to minimize endocytosis) for 15 minutes. Following this very brief incubation, cells were washed and fluorescent images recorded.

Cells incubated with F+ became fluorescently coated (FIG. 1C), while those incubated with F− were not (FIG. 1D). This result indicates that the F+ oligonucleotide was able to direct its base-paired cargo oligonucleotide to the surface of the cells.

### Experimental Example 2

### Targeted Delivery of Long dsRNA

To test whether a similar strategy is effective for delivering long dsRNAs, an experiment was designed to delivery to KB cells dsRNAs to produce siRNAs to cancer-related genes. This experiment addressed both whether tethered complexes can allow cellular uptake of RNAs, as well as whether internalized RNAs are biologically active. Two genes were targeted in this experiment. The first gene, myeloid differentiation factor 88 (MyD88), is a key adaptor protein that is involved in the IL-1R and TLR-induced proinflammatory signaling pathway and that plays an important role in the development of chemoresistant endometrioid ovarian cancer (EOC) cells (Kelly et al., 2006, Cancer Res. 66:3859-3868). The second gene, X-linked inhibitor of apoptosis (XIAP), is a potent apoptosis repressor whose expression is significantly elevated in most chemoresistant EOC cells (Sapi et al., 2004, Oncol Res. 14:567-578). Since suppressed apoptosis is a hallmark of cancer, development of anticancer strategies that specifically target defects in this pathway is highly desirable.

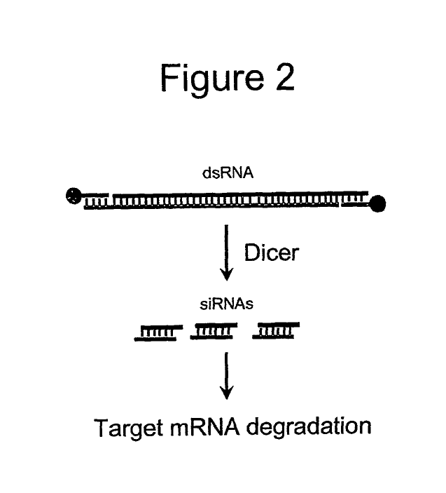

In vitro T7 promoter-based transcription of plasmid DNA clones was used to generate the two strands (sense and antisense) of the dsRNA using a method known in the art (Konze et al., 2002, Nuc. Acids Res. 30:e46). The sense strand of the dsRNA comprises a sequence that is present in the mRNA targeted for RNAi (nucleotides 169-268 of MyD88 mRNA or nucleotides 1261-1500 of XIAP mRNA). Each sense and antisense strand was designed to further comprise a 3′-overhang, having a sequence (hook sequence) that perfectly complements the sequence (hook complement sequence) in SEQ ID NO. 1 shown basepaired in FIG. 1A. As depicted schematically in FIG. 2, dsRNAs specifically targeting MyD88 or XIAP mRNA were separately annealed to molecules of the same F+ODN at both ends. The hypothesis was that upon incubation with KB cells, the F+ODN/dsRNAs would bind to folate receptors on the surface of the KB cells and subsequently be internalized by receptor-mediated uptake. Without being bound by theory, the cellular endonuclease Dicer was expected to process the tethered dsRNA, once inside the cell, into functional siRNAs (FIG. 2).

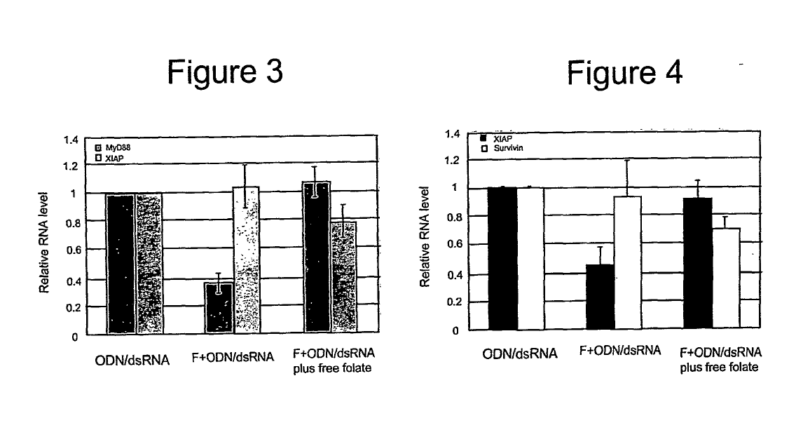

F+ODN/dsRNAs were incubated with KB cells in serum-free culture medium at a final concentration of 100 nM for 2 hours at 37° C. In parallel control experiments, KB cells were incubated with either F+ODN/dsRNA in the presence of 1000-fold molar excess of free folate or with folate-unconjugated ODN/dsRNA. Cells were then washed, fresh medium added, and incubation continued for an additional 22 hours. To assess whether the F+ODN/dsRNAs could specifically reduce the target gene expression in a folate-receptor-dependent manner, total cellular RNAs were extracted, and RNA levels measured using real-time PCR. When the F+ODN/dsRNAs targeting MyD88 was used, a near three-fold reduction in MyD88 mRNA expression was observed (FIG. 3; compare “F+ODN/dsRNA” to “ODN/dsRNA”). Importantly, this reduction could be rescued by the addition of excess free folate (FIG. 3; compare “F+ODN/dsRNA plus free folate” to “F+ODN/dsRNA”). This result supports the premise that delivery is mediated by folate receptors. Moreover, the inhibition of gene expression appeared to be specific, since an mRNA (XIAP) not targeted for RNAi by the dsRNA administered was not significantly affected (FIG. 3). Similar results were obtained when a F+ODN/dsRNA targeted to XIAP mRNA was used (FIG. 4). An approximately two-fold reduction in XIAP mRNA level was obtained (FIG. 4, black bars, compare “F+ODN/dsRNA” to “ODN/dsRNA”). Importantly, this reduction was abolished in the presence of excess free folate (FIG. 4, black bars, compare “F+ODN/dsRNA plus free folate” to “F+ODN/dsRNA”). Moreover, the level of an mRNA not targeted for RNAi (survivin) was not significantly affected with the various treatment (FIG. 4, white bars), further supporting the specific gene silencing effects of the dsRNA. These results strongly suggest that using the tethering method of the invention, dsRNAs can be specifically delivered to folate-receptor-expressing cells and can be processed into siRNAs that generate specific RNAi effects.

### Experimental Example 3

### Targeted Delivery of Long dsRNA

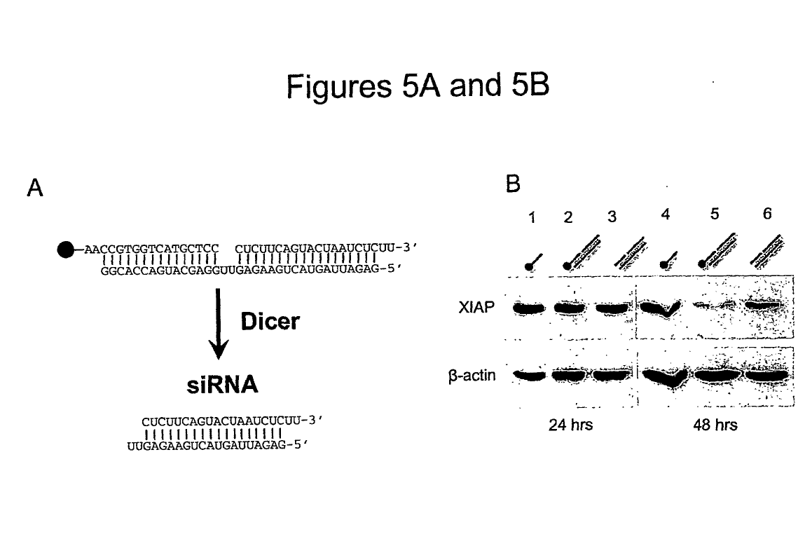

Having demonstrated that siRNA precursors can be delivered to cells using the method of the invention, an experiment was designed to test the effect of siRNA delivery. The siRNA used in the experiment was identical to that used by Sapi et al. (2004, Oncol Res. 14:567-578) to silence XIAP in EOC cells (ovarian cancer cells) with the exception that the sense strand (SEQ ID NO. 3) of the siRNA duplex (comprising SEQ ID NOs. 3 and 4) was extended at its 3′-end to allow base-paired interaction with a tethering oligonucleotide (SEQ ID NO. 5) See FIG. 5A. The annealed complex (F+ODN/XIAP) was added to EOC cells at a final concentration of 200 nM in 10% fetal calf serum. In parallel experiments, cells were incubated with F+ODN alone or with ODN/XIAP. Two hours later, cells were washed and incubated in fresh medium for 24 or 48 hours. For the 48 hour time point, the indicated complexes were added a second time after the first 24 hr of incubation. Total cellular proteins were extracted and levels of XIAP analyzed by Western blotting.

At the 24 hour time point, no significant differences at the XIAP protein level were observed among the samples (FIG. 5B, first three lanes). However, at the 48 hour time point, a nearly three-fold reduction of XIAP was seen with cells treated with F+ODN/XIAP (compare lane 5 to lanes 4 and 6). These data indicate that siRNAs tethered to F+ODNs can be delivered to folate-receptor-expressing cells to induce specific gene silencing. Importantly, since the experiments were performed in the presence of serum, it is believed that the siRNA complexes are reasonably stable against nuclease degradation prior to cellular uptake.

### Experimental Example 4

### Chemoresistant Ovarian Cancer Cells

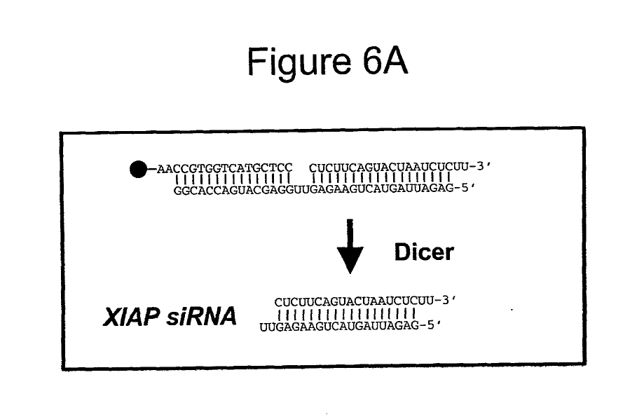

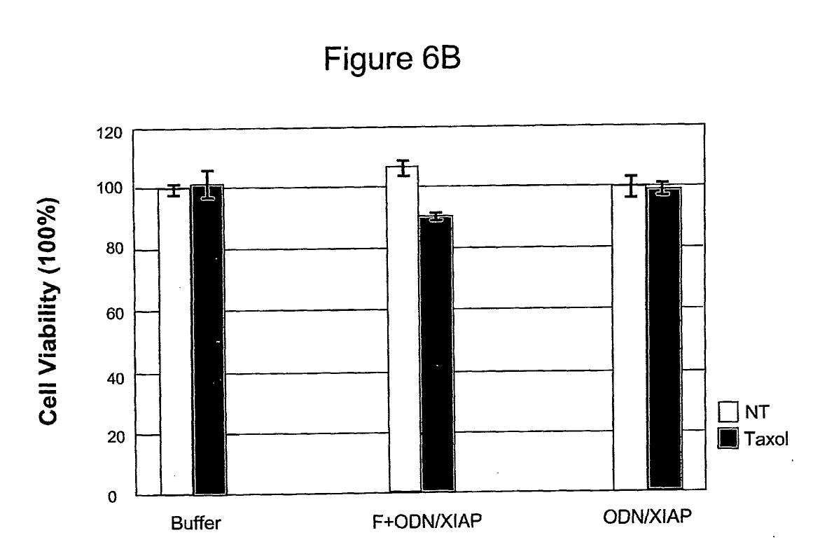

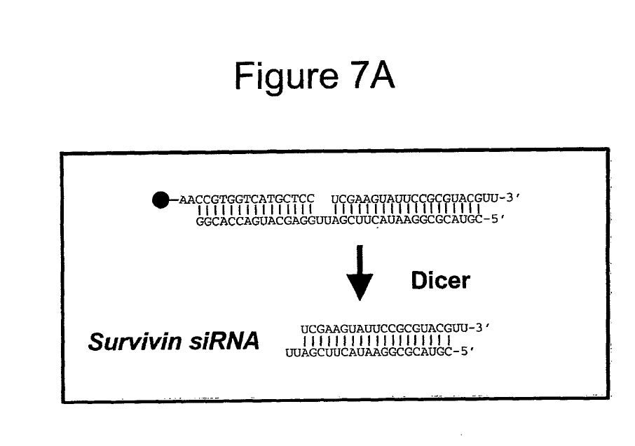

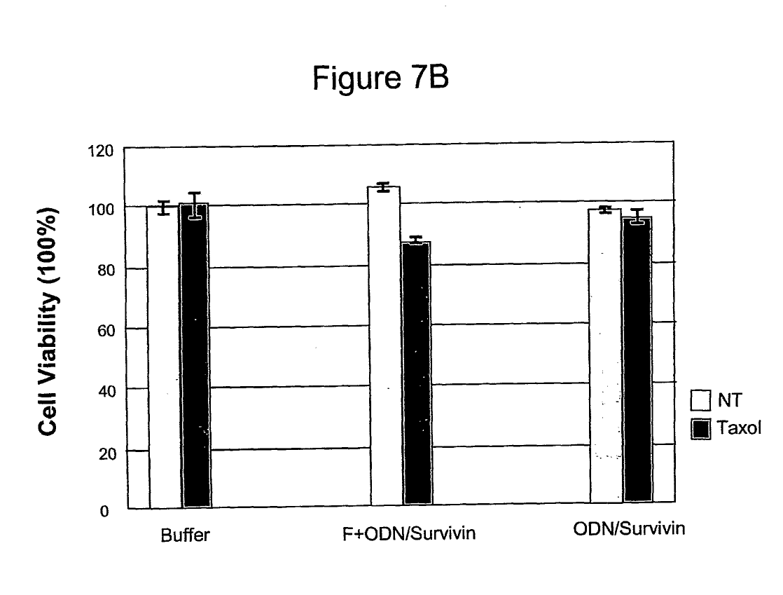

To determine whether reducing the expression of apoptosis-inhibiting genes by siRNAs can restore chemosensitivity to human epithelial ovarian cancer cells (EOC), primary cultures of chemoresistant human EOC cells R182 were incubated with F+ODN/siRNA, ODN/siRNA or buffer alone, at a final concentration of 500 nM in cell culture media for 2 hours. Then, the media were replaced with fresh media that did not contain siRNAs and incubation was continued. Twenty-two (22) hours later, the cells were either treated with the chemotherapy drug Taxol or not treated (NT) for an additional 24 hours, after which cell viability was measured. Cell viability was measured using the CellTiter 96 Aqueous One Solution (Promega, USA) as previously described (Sapi et al., 2004. Oncol Res. 14:567-578). A decreased cell viability indicates chemosensitivity. The two folate-tethered siRNAs tested (F+ODN/XIAP and F+/survivin in FIGS. 6 and 7, respectively) were able to restore chemosensitivity by about 20% in duplicate experiments. See FIGS. 6B and 7B.

### Experimental Example 5

### Targeting Molecule Comprising RGD

A targeting molecule comprising an RGD-containing peptide as the receptor binding moiety is prepared. The RGD-containing peptide has the sequence shown in SEQ ID NO. 6, where the two C-terminal glycine residues serve as a linker. To link to an oligonucleotide, the C-terminal of the peptide has a 6-hydrazino-nicotinamide (HyNic) moiety (SoluLink, San Diego, Calif.), The peptide is conjugated via a disulfide bond an oligonucleotide (SEQ ID NO. 7) to prepare the targeting molecule. Thus, the targeting molecule is the chimeric molecule ACDCRGDCFCGG-CCGTGGTCATGCTCC. The targeting molecule is hybridized to an XIAP siRNA, as shown in FIG. 5A, to prepare a targeted nucleic acid. The targeted nucleic acid is administered to αᵥ,β₃-integrin positive human breast cancer cells. As a negative control, αᵥβ₃-integrin negative human breast cancer cells are also tested. A targeted nucleic acid comprising a non-silencing siRNA (a sequence not known to target any genes in human cells) is prepared to test siRNA specificity.

RGD-dependent cell association (with and without excess unconjugated RGD peptide) is assessed by immunofluorescence. RNA and protein levels are assessed by real time PCR and Western blot, respectively. The RGD-targeted XIAP siRNA complex is expected to reduce RNA levels and protein levels of XIAP in αᵥβ₃-integrin positive human breast cancer cells but is not expected to do so, or to do at a significantly lower level in αᵥβ₃-integrin negative human breast cancer cells.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method for delivering a nucleic acid to a cell, said method comprising:
a) binding non-covalently a targeting molecule to said nucleic acid to form a targeted nucleic acid; and
b) contacting said cell with said targeted nucleic acid;
wherein
i) said targeting molecule comprises a receptor-binding moiety linked to a first oligonucleotide comprising a hook sequence;
ii) said nucleic acid comprises a sequence which is complementary to said hook sequence;
iii) said cell comprises a cognate receptor for said receptor-binding moiety; and
iv) said hook sequence is not a naturally-occurring sequence in said cell;
wherein when said targeted nucleic acid binds to said cognate receptor on said cell, said targeted nucleic acid is taken up by said cell.

2. The method of claim 1, wherein said receptor-binding moiety is selected from the group consisting of folate, an RGD-containing peptide or derivative or peptidomimetics thereof, epidermal growth factor (EGR), transferrin, low density lipoprotein (LDL), insulin, protein hormones, galactosamine, galactose, biotin, platelet-derived growth factor, thyrotrypsin releasing hormone (TRH), nerve-growth factor (NGF), Ct₂-macroglobulin, thiodothyronin, thrombine, arachidonic acid, transforming growth factor-α (TGF-α), a heregulin and alpha fetoprotein (AFP).

3. The method of claim 1, wherein said receptor-binding moiety is wherein said receptor-binding moiety is selected from the group consisting of folate and an RGD-containing peptide or derivative or peptidomimetics thereof.

4. The method of claim 1, wherein said receptor-binding moiety is folate.

5. The method of claim 1, wherein said nucleic acid is selected from the group consisting of dsDNA, antisense, antagomir, siRNA, ribozyme, and miRNA.

6. The method of claim 1, wherein said cell is a human cell.

7. A method for delivering an RNA molecule to a cell, said method comprising:
a) binding a targeting molecule to said RNA molecule to form a targeted RNA molecule; and
b) contacting said cell with said targeted RNA molecule;
wherein
i) said targeting molecule comprises a receptor-binding moiety linked to a first oligonucleotide comprising a hook sequence;
ii) said RNA molecule comprises a sequence which is complementary to said hook sequence; and
iii) said cell comprises a cognate receptor for said receptor-binding moiety;
wherein when said targeted RNA molecule binds to said cognate receptor on said cell, said targeted RNA molecule is taken up by said cell.

8. The method of claim 7, wherein said receptor-binding moiety is selected from the group consisting of folate, an RGD-containing peptide or derivative or peptidomimetics thereof, epidermal growth factor (EGR), transferrin, low density lipoprotein (LDL), insulin, protein hormones, galactosamine, galactose, biotin, platelet-derived growth factor, thyrotrypsin releasing hormone (TRH), nerve-growth factor (NGF), Ct₂-macroglobulin, thiodothyronin, thrombine, arachidonic acid, transforming growth factor-α (TGF-α), a heregulin and alpha fetoprotein (AFP).

9. The method of claim 7, wherein said receptor-binding moiety is selected from the group consisting of folate and an RGD-containing peptide or derivative or peptidomimetics thereof.

10. The method of claim 7, wherein said receptor-binding moiety is folate.

11. The method of claim 7, wherein said RNA molecule is selected from the group consisting of siRNA, ribozyme, and miRNA.

12. The method of claim 7, wherein said cell is a human cell.

13. The method of claim 7, wherein said hook sequence is not a naturally-occurring sequence in said cell.

14. A method for delivering an siRNA molecule to a cell, said method comprising:
a) binding a targeting molecule to said siRNA molecule to form a targeted siRNA molecule; and
b) contacting said cell with said targeted RNA molecule;
wherein
i) said targeting molecule comprises a receptor-binding moiety linked to a first oligonucleotide comprising a hook sequence, wherein said receptor binding moiety is selected from the group consisting of folate and an RGD-containing peptide or derivative or peptidomimetics thereof;
ii) said siRNA molecule comprises a sequence which is complementary to said hook sequence; and
iii) said cell comprises a cognate receptor for said receptor-binding moiety;
wherein when said targeted siRNA molecule binds to said cognate receptor on said cell, said targeted siRNA molecule is taken up by said cell.

15. The method of claim 14, wherein said siRNA molecule is targeted to a gene selected from the group consisting of a gene whose product contributes to the development of chemoresistance and a gene whose product represses apoptosis.

16. The method of claim 14, wherein said cell is a human cell.

17. The method of claim 14, wherein said hook sequence is not a naturally-occurring sequence in said cell.

18. The method of claim 14, wherein said receptor-binding moiety is folate.

19. The method of claim 18, wherein said cell is a human nasopharyngeal carcinoma cell or a human epithelium ovarian carinoma cell.

20. The method of claim 14, wherein said receptor-binding moiety is an RGD-containing peptide or derivative or peptidomimetics thereof.

21. The method of claim 20, wherein said cell is an endothelial cell of neovasculature or a cancer.

22. A composition comprising a targeting molecule bound to a nucleic acid, wherein
i) said targeting molecule comprises a receptor-binding moiety linked to a first oligonucleotide comprising a hook sequence;
ii) said nucleic acid comprises a sequence which is complementary to said hook sequence; and
iii) said hook sequence is not a naturally-occurring sequence.

23. The composition of claim 22, wherein said nucleic acid is selected from the group consisting of siRNA, ribozyme, and miRNA.

24. The composition of claim 22, wherein said receptor-binding moiety is selected from the group consisting of folate, an RGD-containing peptide or derivative or peptidomimetics thereof, epidermal growth factor (EGR), transferrin, low density lipoprotein (LDL), insulin, protein hormones, galactosamine, galactose, biotin, platelet-derived growth factor, thyrotrypsin releasing hormone (TRH), nerve-growth factor (NGF), Ct₂-macroglobulin, thiodothyronin, thrombine, arachidonic acid, transforming growth factor-α (TGF-α), a heregulin and alpha fetoprotein (AFP).

25. The composition of claim 22, wherein said receptor-binding moiety is selected from the group consisting of folate and an RGD-containing peptide or derivative or peptidomimetics thereof.

26. A kit comprising a composition comprising a targeting molecule bound to a nucleic acid and an instructional material, wherein
i) said targeting molecule comprises a receptor-binding moiety linked to a first oligonucleotide comprising a hook sequence;
ii) said nucleic acid comprises a sequence which is complementary to said hook sequence; and
iii) said hook sequence is not a naturally-occurring sequence.

27. The kit of claim 26, wherein said nucleic acid is selected from the group consisting of siRNA, ribozyme, and miRNA.

28. The kit of claim 26, wherein said receptor-binding moiety is selected from the group consisting of folate, an RGD-containing peptide or derivative or peptidomimetics thereof, epidermal growth factor (EGR), transferrin, low density lipoprotein (LDL), insulin, protein hormones, galactosamine, galactose, biotin, platelet-derived growth factor, thyrotrypsin releasing hormone (TRH), nerve-growth factor (NGF), Ct₂-macroglobulin, thiodothyronin, thrombine, arachidonic acid, transforming growth factor-α (TGF-α), a heregulin and alpha fetoprotein (AFP).

29. The composition of claim 26, wherein said receptor-binding moiety is selected from the group consisting of folate and an RGD-containing peptide or derivative or peptidomimetics thereof.

30. A kit comprising:
a) a targeting molecule, wherein said targeting molecule comprises a receptor-binding moiety linked to a second oligonucleotide comprising a hook sequence;
b) a second oligonucleotide comprising a hook complement sequence; and
c) an instructional material,
wherein said hook sequence is not a naturally-occurring sequence.
